(19) United States
(12) Patent Application Publication
(10) Pub. No.: US 2023/0158190 A1
(43) Pub. Date: May 25, 2023

(54) Apparatus for Disinfecting Objects or Solids, Preferably Pieces of Protective Equipment, and Its Use

(71) Applicant: PlasmaTreat GmbH, Steinhagen (DE)

(72) Inventors: Thomas Schmitt-John, Steinhagen (DE); Daniel Hasse, Paderborn (DE); Roland Heinrich Kläsener, Schloss Holte Stuckenbrock (DE); Christian Buske, Bielefeld (DE)

(21) Appl. No.: 17/913,600

(22) PCT Filed: Mar. 18, 2021
(86) PCT No.: PCT/EP2021/056920

§ 371 Date: Sep. 22, 2022

(30) Foreign Application Priority Data
Mar. 23, 2020 (DE) 10 2020 107 981.8

(51) Int. Cl. A61L 2/20; A61L 2/26; A61L 2/14

(52) U.S. Cl. CPC A61L 2/202; A61L 2/14; A61L 2/26; A61L 2202/11; A61L 2202/13; A61L 2202/26; A61L 2202/121; A61L 2202/122; A61L 2202/123

(57) ABSTRACT

The invention relates to an apparatus for disinfecting, in particular sterilizing, objects or solids, preferably pieces of protective equipment, in particular protective masks or protective clothing, with a treatment chamber for receiving one or more objects or solids, preferably one or more pieces of protective equipment, with a closable airlock through which objects or solids, preferably pieces of protective equipment, can be introduced into the treatment chamber and/or removed from the treatment chamber and with a generation unit for generating reactive species in a gas stream. The generation unit includes discharge means configured to generate an electrical discharge in the gas stream. The generating unit is arranged such that the gas stream, during operation, passes from the generating unit into the treatment chamber. The invention further relates to a use of the apparatus.

### CROSS-REFERENCE TO RELATED APPLICATIONS

This application is the U.S. National Phase of International Application No. PCT/EP2021/056920 filed Mar. 18, 2021, and claims priority to German Patent Application No. 10 2020 107 981.8 filed Mar. 23, 2020, the disclosures of which are hereby incorporated by reference in their entirety.

### BACKGROUND OF THE INVENTION

### Field of the Invention

The present invention relates to an apparatus for disinfecting, in particular for sterilizing, objects or solids, preferably pieces of protective equipment, in particular protective masks or protective clothing.

### Description of Related Art

To protect against pathogens such as bacteria, viruses or spores, protective masks or protective clothing such as protective suits are used in particular. Such pieces of protective equipment can typically only be used for a limited period of time before they need to be changed. For example, depending on the type, protective masks may need to be changed daily, after a few hours, hourly or even at shorter intervals.

After use, the protective equipment must be laboriously disinfected or, in the case of disposable protective equipment, disposed of directly.

In times of increased demand or shortage of supplies, there may be an undersupply of protective equipment.

There is a need to disinfect protective equipment quickly and easily so that it can be made available for use again more quickly. Furthermore, there is a need to be able to use for a longer period of time or to reuse protective equipment that is otherwise only suitable for single use.

### SUMMARY OF THE INVENTION

Based on this, the present object is to propose a solution for at least partially satisfying at least one of the aforementioned needs.

This object is solved according to the invention by an apparatus for disinfecting, in particular for sterilizating, objects or solids, preferably of pieces of protective equipment, in particular protective masks or protective clothing, with a treatment chamber for receiving one or more objects or solids, preferably one or more pieces of protective equipment, with a closable airlock through which objects or solids, preferably pieces of protective equipment, can be introduced into the treatment chamber and/or removed from the treatment chamber, and with a generation unit for generating reactive species in a gas stream, wherein the generation unit comprises discharge means arranged to generate an electrical discharge in the gas stream, and wherein the generation unit is arranged such that the gas stream, during operation, passes from the generation unit into the treatment chamber.

With such an apparatus, objects or solids can be disinfected quickly, easily and reliably. In particular, the objects or solids may be one or more goods. In particular, the objects may be one or more pieces of one or more goods.

The apparatus described above can be used to disinfect, in particular sterilize, protective equipment quickly, easily and reliably in order to make it ready for a new use. Furthermore, such an apparatus can also be used, for example, to disinfect items of protective equipment such as breathing masks, which are otherwise only intended for single use. In this way, the service life of the protective equipment can be extended and the protective equipment can be reused as far as permissible.

In addition to the disinfection of protective equipment, the apparatus can also be used for the disinfection of other goods such as powders, seeds or foodstuffs, in particular vegetables, fruit, lettuce, nuts such as hazelnuts, almonds, pulses or spices such as pepper.

The apparatus has a treatment chamber. The treatment chamber is preferably completely closable except for inlets and outlets required for operation, so that no reactive species can escape uncontrolled from the treatment chamber during operation. In particular, the treatment chamber is dimensioned to accommodate one or more objects or solids, preferably one, particularly several pieces of protective equipment, in particular one or more breathing masks and/or pieces of protective clothing.

The apparatus further comprises a closable airlock through which objects or solids, preferably pieces of protective equipment, can be introduced into the treatment chamber and/or removed from the treatment chamber. The airlock may be, in particular, a door provided in a side wall of the treatment chamber which, when open, allows access to the treatment chamber so that objects or solids, preferably pieces of protective equipment, can be introduced into the treatment chamber and/or removed from the treatment chamber, and which, when closed, closes the treatment chamber.

The apparatus further comprises a generation unit for generating reactive species in a gas stream. Accordingly, the generation unit is particularly configured to generate reactive species in a gas stream. To this end, the generation unit comprises discharge means which are configured to generate an electrical discharge in the gas stream. In particular, the discharge means may comprise electrodes to which a high frequency high voltage may be applied to generate electrical discharges in the gas stream. Furthermore, the discharge means may comprise a voltage source, for example a transformer, for applying a high-frequency high voltage to the electrodes.

The electrical discharge causes the formation of reactive species in the gas stream. In particular, the reactive species may be one or more of the following: ozone, nitrogen oxides, hydroxyl radicals, nitrites, nitrates, fully or partially ionized or excited atoms or molecules. The gas stream may be at least partially converted to the plasma state by the electrical discharge.

According to the invention, the above-mentioned object is further solved by the use of the previously described apparatus for disinfecting, in particular sterilizing, objects or solids. Preferably, the apparatus is used for disinfecting, in particular sterilizing, protective equipment, in particular protective masks, such as FFP2 or FFP3 masks, or protective clothing (e.g. Tychem or Mikrogard). Furthermore, the apparatus can be used for disinfecting, in particular sterilizing, other goods, in particular powders, seeds or foodstuffs, in particular vegetables, fruit, lettuce, nuts such as hazelnuts, almonds, pulses or spices such as pepper.

Various embodiments of the apparatus and of the use are described below, with the individual embodiments applying individually to both the apparatus and the use. Furthermore, the individual embodiments may be combined with each other.

In one embodiment, the discharge means are configured to generate a dielectric barrier discharge in the gas stream. By means of a dielectric barrier discharge, very high concentrations of certain reactive species, in particular ozone, can be generated in the gas stream, whereby a strong disinfecting, in particular sterilizing effect is achieved.

The discharge means for producing a dielectric barrier discharge may in particular comprise at least two electrodes and a dielectric disposed therebetween which impedes a direct electrical discharge between the two electrodes. Preferably, one of the electrodes is grounded. Furthermore, the discharge means may in particular comprise a voltage source for applying a high-frequency high voltage to the electrodes, for example with a voltage amplitude in the range of 5 to 15 kV and a voltage frequency in the range of 7.5 to 25 kHz, in particular 13 to 14 kHz.

In one embodiment, the discharge means are configured to generate a high-frequency high-voltage discharge, in particular between at least two electrodes, in the gas stream. Preferably, the discharge means are configured for generating an arc-like discharge in a gas stream, wherein the arc-like discharge is generated by applying a high-frequency high voltage between electrodes.

In particular, the generation unit can comprise a plasma nozzle for generating an atmospheric plasma jet, through which plasma nozzle the gas stream flows during operation, the plasma nozzle having discharge means in the form of electrodes between which a high-frequency high-voltage can be applied via a high-voltage source configured for this purpose, so that high-frequency high-voltage discharges occur, in particular a high-frequency arc-like discharge, by means of which the gas stream passed through the plasma nozzle is enriched with reactive species.

In this way, a high concentration of certain reactive species can be generated in the gas stream, in particular nitrogen oxides and/or fully or partially ionized or excited atoms or molecules. Ozone is generated only to a small extent compared to dielectric barrier discharges, thus reducing the ozone load. In contrast, nitrogen oxides are increasingly generated by a high-frequency arc-like discharge.

A high-frequency high voltage, in particular for generating a high-frequency arc-like discharge, is understood to mean in particular a voltage of 1 - 100 kV, preferably 1 - 50 kV, more preferably 10 - 50 kV, at a frequency of 1 - 300 kHz, in particular 1 - 100 kHz, preferably 10 - 100 kHz, more preferably 10 - 50 kHz.

In a further embodiment, the generation unit has a first part generation unit and a second part generation unit, the first part generation unit comprising first discharge means for generating a dielectric barrier discharge in a first partial gas stream, and the second part generation unit comprising discharge means for generating a high-frequency high-voltage discharge in a second partial gas stream. Preferably, the generation unit is configured to combine, in particular to mix, the first partial gas stream and the second partial gas stream. The merging, in particular mixing, preferably takes place before the partial gas streams are supplied to the treatment chamber. It was found that the first part generation unit generates reactive species in the first partial gas stream with very high efficiency and concentration. Furthermore, it was found that the ozone generated with the first part generation unit can be partially or completely annihilated by the second partial gas stream from the second part generation unit. In this way, the ozone load can be reduced while the common gas stream created by mixing the first and second partial gas streams continues to have a sterilizing effect.

In a further embodiment, the generating unit is arranged within the treatment chamber. In this way, the distance between the discharge means and the objects or solids, preferably pieces of protective equipment, arranged in the treatment chamber during operation is kept as small as possible, whereby a high disinfection effect is achieved.

In a further embodiment, the generation unit is integrated into the wall of the treatment chamber. In particular, an outlet of the generation unit, from which the gas stream with the reactive species exits during operation, can be integrated into the wall of the treatment chamber. In this way, the distance between the discharge means and the objects or solids, preferably pieces of protective equipment, arranged in the treatment chamber during operation can be kept small, while at the same time the generating unit is largely protected from the atmosphere in the treatment chamber, thus extending its service life.

In another embodiment, the generation unit is arranged outside the treatment chamber and is connected to the treatment chamber in such a way that the gas stream enters the treatment chamber during operation. In this way, the generation unit can be positioned more flexibly. In addition, the spatial arrangement can in this way already reliably prevent a user from coming into contact with the discharge means of the generation unit, thus increasing operational safety.

In one embodiment, a fan is arranged in the treatment chamber. In this way, the gas stream from the generation unit can be better distributed in the treatment chamber during operation, so that objects or solids arranged in the treatment chamber, preferably pieces of protective equipment, are disinfected more uniformly.

In a further embodiment, one or more positioning devices are arranged in the treatment chamber for positioning objects or solids, preferably pieces of protective equipment, at a predetermined position in the treatment chamber. In this way, it can be ensured that the objects or solids, preferably pieces of protective equipment, are arranged in the treatment chamber in such a way that a most uniform and complete disinfection of the objects or solids, preferably pieces of protective equipment, is achieved. For example, one or more hooks or other holding devices for breathing masks or pieces of protective clothing can be provided as a positioning device, which are arranged at predetermined positions within the treatment chamber.

In a further embodiment, a tubular element is arranged in the treatment chamber in such a way that, during operation, the gas stream introduced into the treatment chamber flows through it, the tubular element being configured to receive a plurality of objects, preferably items of protective equipment, in particular a plurality of breathing masks, in such a way that the objects, preferably items of protective equipment, in particular breathing masks, are flowed through by the gas stream as it flows through the tubular element, in particular one after the other. It has been found that objects, in particular pieces of protective equipment such as breathing masks, can be disinfected more effectively when they are forcibly flowed through with a gas stream containing reactive species. By providing the tubular element, the gas stream is passed through the objects arranged therein, preferably pieces of protective equipment, in particular breathing masks, whereby a substantially better disinfection can be achieved than by diffusely flowing gas containing reactive species around the objects, in particular pieces of protective equipment, in particular breathing masks. In particular, the tubular element can be configured to accommodate a number of objects, preferably items of protective equipment, in particular breathing masks, arranged one behind the other with respect to the direction of flow.

In a further embodiment, a perforated plate is arranged in the treatment chamber in such a way that the gas stream introduced into the treatment chamber flows through it during operation, the perforated plate being arranged in particular in such a way that a number of objects, preferably a plurality of items of protective equipment, in particular a plurality of breathing masks, can be positioned on the perforated plate. In this way, the perforated plate represents a simple positioning device for objects, preferably pieces of protective equipment, in particular breathing masks. It has been found that by providing a perforated plate for the arrangement of objects, preferably pieces of protective equipment, in particular breathing masks, a targeted flow of the gas stream through the objects, preferably pieces of protective equipment, can be achieved.

Preferably, a suction device, in particular comprising a fan, is provided to suck the gas stream through the perforated plate. In this way, the gas stream can be directed through the objects positioned on the perforated plate, preferably pieces of protective equipment, with a higher throughput.

In a further embodiment, an outlet is provided on the treatment chamber for discharging the gas stream from the treatment chamber. In this way, gas, in particular gas with reactive species, can be discharged from the treatment chamber during or at the end of a disinfection process, so that the exposure of the user to reactive species, in particular ozone, is reduced when the airlock is opened. For this purpose, exhaust means can preferably be connected to the outlet, which are adapted to exhaust the gas stream from the treatment chamber. In this way, gas containing reactive species can be selectively removed from the treatment chamber. Preferably, a fresh air inlet is provided at the treatment chamber, through which fresh air can flow in particular at the end of a disinfection process when the gas stream is discharged, in particular sucked off, from the treatment chamber.

In a further embodiment, a neutralization device is provided at the outlet, which is arranged to reduce the ozone content of the gas stream, in particular of the gas stream discharged from the treatment chamber. In this way, the ozone pollution of the environment can be reduced.

In particular, the neutralization device may comprise a plasma nozzle for generating an atmospheric plasma jet. It has been found that the ozone content in the gas stream can be significantly reduced with an atmospheric plasma jet. The plasma nozzle is preferably configured to generate a plasma jet by high-frequency, high-voltage discharges in a working gas stream. A plasma jet generated in this way reduces ozone very effectively. To reduce the ozone content in the gas stream discharged from the treatment chamber, the gas stream can be passed through the plasma nozzle, in particular as a working gas stream. Alternatively, it is also conceivable to impinge the gas stream discharged from the treatment chamber with the plasma jet emerging from the plasma nozzle.

In a further embodiment, a recirculation system is provided which is configured to discharge the gas stream from the treatment chamber and to resupply it to the treatment chamber via a recirculation duct system. In this way, reactive species can be repeatedly generated in the gas stream so that an overall higher concentration of reactive species is achieved in the gas stream. In addition, the circulation of the gas stream brought about by the recirculation system can achieve a better distribution of the gas stream with the reactive species in the treatment chamber. In particular, the recirculation system comprises at least one recirculation inlet at the treatment chamber, through which the gas stream from the treatment chamber reaches the recirculation duct system, and at least one recirculation outlet at the treatment chamber, through which the gas stream from the recirculation duct system reaches the treatment chamber again.

In a further embodiment, the recirculation system has a fan, in particular a side-channel compressor, which fan is configured to extract the gas stram from the treatment chamber and to direct it through the recirculation duct system. In this way, a controllable gas stream is ensured in the recirculation system. In particular, the fan can be arranged in the recirculation duct system.

In a further embodiment, the generation unit is integrated into the recirculation system in such a way that the gas stream conducted through the recirculation system is at least partially supplied to the generation unit. In this way, the circulated gas stream, which typically still contains some reactive species, is further enriched with reactive species by the generation unit. The generation unit may be located between a first and a second section of the recirculation duct system, the first section directing the gas stream from the treatment chamber to the generation unit and the second section directing the gas stream from the generation unit back to the treatment chamber. Alternatively, the generation unit may be arranged at the end of the recirculation duct system so that the gas stream from the generation unit enters the treatment chamber. It is further conceivable that the generation unit is arranged in a bypass line branching off from a main line of the recirculation duct system, the bypass line joinung again into the main line or directly into the treatment chamber on the downstream side of the generation unit.

In a further embodiment, the generation unit is formed separately from the recirculation system. Preferably, for this purpose, the generation unit has a supply line separate from the recirculation system for supplying the generation unit with a separate gas stream, for example with a separate fresh air supply. By separating the recirculation system and the generation unit, the service life of the generation unit can be improved, as it has less contact with the reactive species in the circulated gas stream.

In a further embodiment, humidifying means are provided which are configured to increase the relative humidity of the gas stream and/or the relative humidity in the treatment chamber, preferably to a relative humidity in the range of 90% RH - 100% RH, preferably 95% RH - 98% RH. It has been found that by increasing the relative humidity, especially in the above ranges, in combination with the reactive species in the gas stream, a better disinfection effect can be obtained. In particular, the relative humidity of the gas stream is increased before it enters the area of the treatment chamber intended for the objects or solids, preferably pieces of protective equipment.

In a further embodiment, the humidifying means comprise a preferably heatable water trough. In this way, the advantageous increase in relative humidity can be effected in a simple manner. The water trough may in particular be arranged in the treatment chamber. However, it is also conceivable to arrange the water trough outside the treatment chamber, in particular if the generation unit is arranged outside the treatment chamber.

However, it has been found that an increase in the relative humidity in the gas stream can already be achieved with an unheated water trough, especially with water at room temperature. Therefore, in order to save costs, for example, heating agents can be dispensed with.

However, in order to achieve a higher evaporation performance, heating means are preferably provided for heating the water trough, which are particularly preferably configured to feed-back control water contained in the water trough to a predetermined set temperature, in particular in the range of 50 - 100° C., preferably 50 - 80° C. In this way, more water vapor can be generated and the relative humidity of the gas stream can be increased more effectively, for example, even at higher gas stream flow rates.

In a further embodiment, the water trough is arranged such that the gas stream from the generating unit is directed onto the water trough during operation. For this purpose, for example, a conduit or a nozzle orifice may be provided through which the gas stream is directed onto the water trough during operation. It has been found that the relative humidity of the gas stream can be increased very easily and effectively by blowing the gas stream over the water surface of the liquid water in the water trough. In this way, the water vapor above the water surface accumulates in the gas stream.

In a further embodiment, an evaporative body is arranged in the water trough. In this way, the gas stream can be humidified more effectively. On the one hand, a higher evaporation performance can be achieved with such an evaporation body. On the other hand, such an evaporation body favors that small water droplets are entrained with the gas stream, so that the water content of the gas stream is increased in this way. The evaporation body preferably consists of a porous, in particular sponge-like material. Such materials have a very large surface area in relation to their volume, which improves the humidification of the gas stream. The evaporation body is preferably arranged in such a way that the gas stream flows around and/or through it during operation.

In a further embodiment, a nebulizer, in particular an ultrasonic nebulizer, is arranged in the water trough. In this way, the evaporation performance can also be improved.

In a further embodiment, the water trough is filled with plasma-activated water. In this way, the disinfection effect can be further improved.

Plasma-activated water is understood to mean water that has been activated by exposure to a working gas emerging from an atmospheric plasma source. In particular, the water can be directly exposed to atmospheric plasma, such as an atmospheric plasma jet, that is, to a working gas emerging from the plasma source that is still at least partially in the plasma state. Furthermore, the water can also be exposed to the working gas exiting the plasma source after the working gas has already recombined, i.e. is no longer in the plasma state. It has been found that even in such a recombined working gas there are still sufficient reactive species, for example ozone or nitrogen oxides, which form relatively long-living reactive species in the water, such as hydrogen peroxide, nitric acid or nitrous acid.

Accordingly, the plasma-activated water may be or may have been produced by exposure of (in particular liquid) water to a working gas emanating from an atmospheric plasma source.

Suitable plasma-activated water and a device for its production are described, for example, in EP 3 470 364 A1.

When using plasma-activated water, it is particularly advantageous to provide an evaporator or a nebulizer in the water trough, because these promote the entrainment of water droplets with the gas stream. Compared to pure evaporation of the water, moistening the gas stream with water droplets has the advantage that the reactive species from the plasma-activated water enter the gas stream to a greater extent.

### BRIEF DESCRIPTION OF THE DRAWINGS

Further features and advantages of the apparatus and the use will be apparent from the following description of embodiments, reference being made to the accompanying drawing.

In the drawing FIG. 1 shows a plasma nozzle for generating an atmospheric plasma jet,

FIG. 2 shows a nozzle for generating reactive species in a gas stream by means of dielectric barrier discharge,

FIG. 3 shows an exemplary embodiment of the apparatus for disinfecting objects or solids, preferably pieces of protective equipment, and for its use,

FIG. 4 shows the generation unit of the apparatus from FIG. 3,

FIG. 5 shows an alternative generation unit for the apparatus in FIG. 3,

FIG. 6 shows another alternative generation unit for the apparatus in FIG. 3,

FIG. 7 shows another alternative generation unit for the apparatus in FIG. 3,

FIG. 8 shows a further examplary embodiment of the apparatus for disinfecting objects or solids, preferably pieces of protective equipment, and for its use,

FIG. 9 shows the neutralization unit of the apparatus in FIG. 8,

FIG. 10 shows an alternative neutralization unit for the apparatus in FIG. 8,

FIG. 11 shows another examplary embodiment of the apparatus for disinfecting objects or solids, preferably pieces of protective equipment, and for its use,

FIG. 12 shows a further examplary embodiment of the apparatus for disinfecting objects or solids, preferably pieces of protective equipment, and for its use,

FIG. 13 shows a further exemplary embodiment of the apparatus for disinfecting objects or solids, preferably pieces of protective equipment, and for its use,

FIG. 14 shows a further exemplary embodiment of the apparatus for disinfecting objects or solids, preferably pieces of protective equipment, and for its use,

FIG. 15 shows a further exemplary embodiment of the apparatus for disinfecting objects or solids, preferably pieces of protective equipment, and for its use,

FIGS. 16a-b show a further exemplary embodiment of the apparatus for disinfecting objects or solids, preferably pieces of protective equipment, and for its use,

FIG. 17 shows a further alternative generating unit for the apparatus in FIG. 3 and

FIG. 18 shows a further alternative generating unit for the apparatus in FIG. 3.

### DESCRIPTION OF THE INVENTION

FIG. 1 shows a plasma nozzle 2 for generating an atmospheric plasma jet 26.

The plasma nozzle 2 has a nozzle tube 4 made of metal, which tapers conically to a nozzle opening 6. At the end opposite the nozzle opening 6, the nozzle tube 4 has a swirl device 8 with an inlet 10 for a gas stream, in particular a working gas, for example nitrogen.

An intermediate wall 12 of the swirl device 8 has a ring of bores 14 set obliquely in the circumferential direction, through which the gas stream is twisted. The downstream, conically tapered part of the nozzle tube is therefore flowed through by the gas stream in the form of a vortex 16, the core of which runs along the longitudinal axis of the nozzle tube. An electrode 18 is arranged centrally on the underside of the intermediate wall 12, which projects coaxially into the nozzle tube in the direction of the tapered section. The electrode 18 is electrically connected to the intermediate wall 12 and the other parts of the swirl device 8. The swirl device 8 is electrically insulated from the nozzle tube 4 by a ceramic tube 20. A high-frequency high voltage, which is generated by a transformer 22, is applied to the electrode 18 via the swirl device 8. The inlet 10 is supplied with a gas stream 23 via a line not shown. The nozzle tube 4 is grounded. The applied voltage generates a high frequency discharge in the form of an arc 24 between the electrode 18 and the nozzle tube 4. The electrode 18 connected to the transformer and the grounded nozzle pipe 4 thus constitute discharge means 25 which are configured to generate a high-frequency high-voltage discharge in the form of the arc 24 in a gas stream 23.

The terms “arc”, “arc discharge” or “arc-like discharge” are used here as a phenomenological description of the discharge, since the discharge occurs in the form of an arc. The term “arc” is also used elsewhere as a form of discharge for DC discharges with essentially constant voltage values. However, the present case involves a high-frequency discharge in the form of an arc, i.e. a high-frequency, arc-like discharge.

Due to the swirling flow of the working gas, however, this arc is channeled in the vortex core on the axis of the nozzle tube 4, so that it branches out to the wall of the nozzle tube 4 only in the region of the nozzle opening 6. The working gas, which rotates at high flow velocity in the region of the vortex core and thus in the immediate vicinity of the arc 24, comes into intimate contact with the arc and is thereby partially converted into the plasma state, so that an atmospheric plasma jet 26 emerges from the plasma nozzle 2 through the nozzle opening 6.

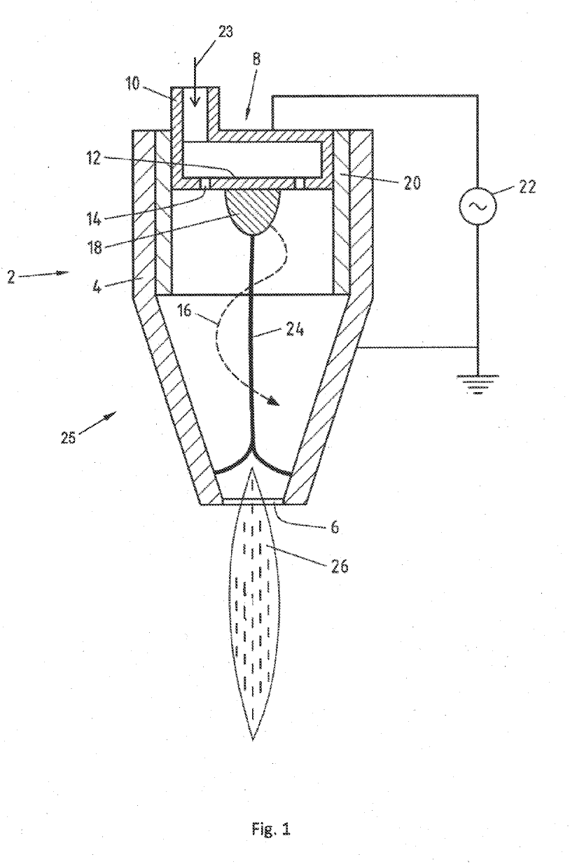

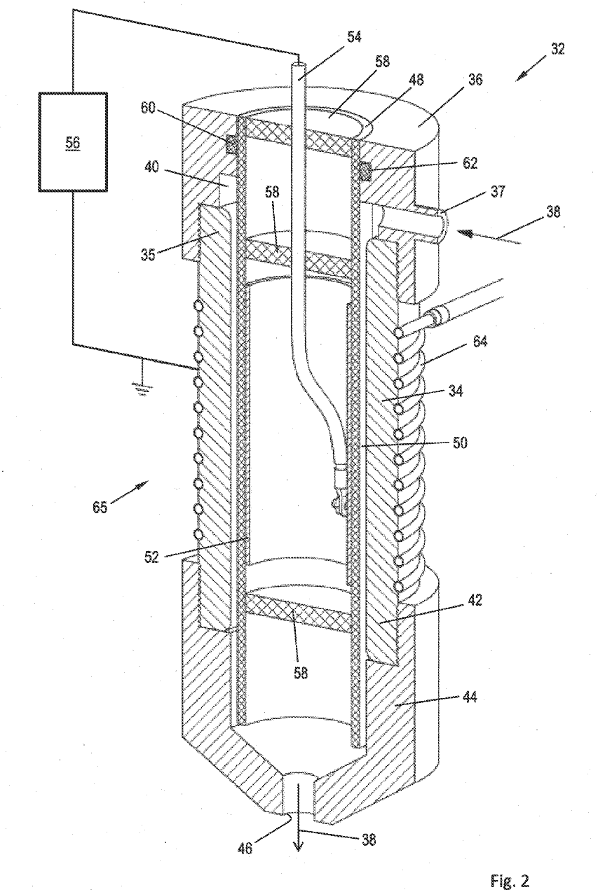

FIG. 2 shows a nozzle for generating reactive species in a gas stream by means of dielectric barrier discharge.

The nozzle 32 has a nozzle tube 34 made of metal, at the upstream end 35 of which a distributor head 36 with an inlet 37 for a gas stream 38, in particular for a working gas stream, and with an annular distributor channel 40 is arranged. An outlet nozzle 44 with a nozzle opening 46 is arranged at the opposite downstream end 42 of the nozzle tube 34, from which nozzle opening the gas stream 38 enriched with reactive species emerges during operation.

From the distributor head 36, a ceramic tube 48 extends through the nozzle tube 34 into the outlet nozzle 44 in such a way that an annular discharge channel 50 extends from the distributor channel 40 between the nozzle tube 34 and the ceramic tube 48 to the outlet nozzle 44. Instead of a ceramic tube, for example, a tube made of quartz glass could also be considered.

A tubular high-voltage electrode 52 made of metal is arranged on the inside of the ceramic tube 48, which is connected to a transformer 56 via a high-voltage cable 54, with which a high-frequency high voltage can be applied between the high-voltage electrode 52 and the grounded nozzle tube 34 acting as a counter-electrode. Instead of a tubular high-voltage electrode 52, for example, a differently shaped high-voltage electrode could also be considered, for example in the form of a rounded sheet.

Insulating plugs 58 are disposed in the ceramic tube 48 to enclose the high voltage electrode 52 and further prevent working gas from flowing into the area of the high voltage electrode 52 or flowing out of the nozzle 32 through the ceramic tube 48. Further, a sealing ring 60 is inserted into an annular groove 62 on the manifold head 36 to seal the manifold head 36 to the ceramic tube 48.

A coolant line 64 may be provided around the nozzle tube 34, through which a coolant may be passed during operation to cool the nozzle tube 34. The coolant line 64 can, for example, run spirally around the nozzle tube 34 as shown.

In operation, a gas stream 38 is introduced into the manifold head 36 through the inlet 37 so that the gas stream 38 flows through the annular discharge channel 50.

The transformer 56 is used to apply a high-frequency high voltage between the high-voltage electrode 52 and the nozzle tube 34, so that dielectric barrier discharges occur in the discharge channel 50 in the region of the high-voltage electrode 52, as a result of which reactive species, in particular ozone, are generated in the gas stream 38 flowing there. The high-voltage electrode 52 connected to the generator 56 and the grounded nozzle tube 34 thus represent discharge means 65 which are configured to generate a dielectric barrier discharge in a gas stream 38.

The gas stream 38 enriched with the reactive species exits the nozzle orifice 46.

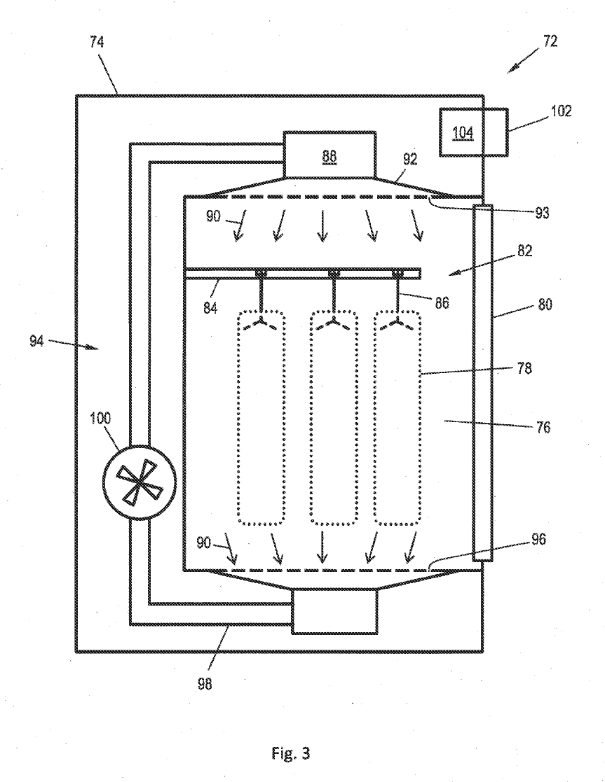

FIG. 3 shows an exemplary embodiment of the apparatus for disinfecting objects or solids, preferably protective equipment parts, and for its use.

The apparatus 72 has a housing 74 in which a treatment chamber 76 is arranged for receiving pieces of protective equipment, such as protective masks or protective clothing 78. A closable airlock 80 in the form of a door attached to the housing 74 is provided on one side of the treatment chamber 76, through which a user can arrange items of protective equipment to be disinfected in the treatment chamber 76 or remove the disinfected items of protective equipment from the treatment chamber 76 at the end of the disinfection process.

Positioning devices 82 are provided in the treatment chamber 76 for positioning pieces of protective equipment at predetermined locations in the treatment chamber 76. In the present exemplary embodiment, the positioning devices 82 are in the form of a holder 84 with a plurality of hangers 86 for hanging protective clothing 78. In this way, wrinkling of the protective garments 78 can be prevented and the protective garments 78 have a predetermined spacing from each other, allowing for more uniform and reliable disinfection.

The apparatus 72 further comprises a generation unit 88 for generating reactive species in a gas stream 90. The generation unit 88 is disposed above the treatment chamber 76 and is connected thereto via a manifold 92 having a plurality of openings 93, through which the gas stream 90 enriched with reactive species by the generation unit 88 flows into the treatment chamber 76 during operation and causes disinfection of the protective equipment disposed in the treatment chamber 76.

The generation unit 88 is integrated into a recirculation system 94, which has recirculation inlets 96 at the bottom of the treatment chamber 76, through which the gas stream 90 passes into a recirculation duct system 98 after passing through the treatment chamber 76. A fan 100, in particular a side channel compressor, is arranged in the recirculation duct system 98, which sucks the gas stream 90 out of the treatment chamber 76 through the recirculation air inlets 96 and feeds it back to the generation unit 88.

Since the gas stream 90 typically still contains reactive species after passing through the treatment chamber 76, the recirculation system 94 can be used to successively increase the reactive species in the gas stream 90, thereby improving the disinfection effect.

The apparatus 72 may further comprise a control unit 102 connected to a control device 104, via which a user may operate the apparatus 72. The control device 104 is configured to control the various components of the apparatus 72, in particular the generation unit 88 and the fan 100, depending on user input received via the control unit 102.

To use the apparatus 72 to disinfect protective equipment items, the user first opens the airlock 80 and positions the protective equipment items to be disinfected, for example the protective clothing items 78 shown in FIG. 3, using the positioning devices 82 provided for this purpose. The user then closes the airlock 80 and activates the apparatus 72 via the control unit 102. The control device 104 then controls the generation unit 88 and the fan 100 in such a way that the generation unit 88 generates reactive species in the gas stream 90, which is guided by the fan through the treatment chamber 76 and the recirculation system 94. After a predetermined time has elapsed, the control device deactivates the generation unit 88 and the fan 100. The user can then remove the disinfected pieces of protective equipment from the treatment chamber 76 after opening the airlock 80.

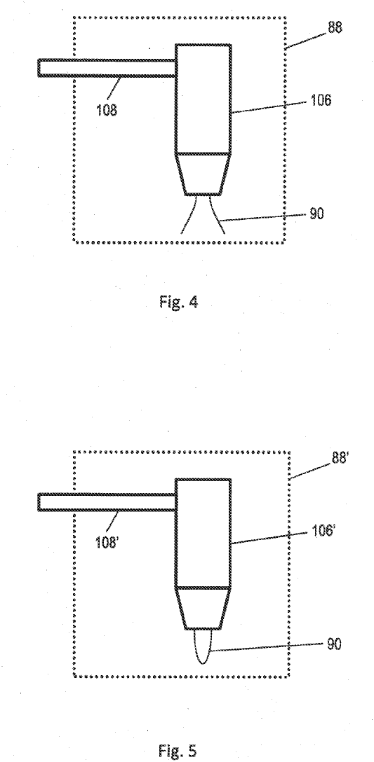

FIG. 4 shows the generation unit 88 of the apparatus 72 of FIG. 3. The generation unit 88 comprises a nozzle 106 for generating reactive species in the gas stream 90 passed through the nozzle 106, the nozzle 106 having discharge means configured to generate a dielectric barrier discharge in the gas stream 90 supplied to the nozzle 106 via a supply line 108. In particular, the nozzle 106 may be configured like the nozzle 32 shown in FIG. 2. In the apparatus 72 of FIG. 3, the supply line 108 is connected to the recirculation duct system 98, so that the gas stream 90 extracted from the treatment chamber 76 is supplied to the nozzle 106 again. The use of discharge means to produce a dielectric barrier discharge results in a gas stream 90 with a high ozone concentration and thus a good disinfection effect.

FIG. 5 shows an alternative generation unit 88′ that can be used in place of the generation unit 88 for the apparatus of FIG. 3. The generation unit 88′ comprises a nozzle 106′ for generating reactive species in the gas stream 90, the nozzle 106′ having discharge means arranged to generate a high-frequency, high-voltage discharge in the gas stream 90 supplied to the nozzle 106′ via the supply line 108′. In particular, the nozzle 106′ may be configured like the plasma nozzle 2 shown in FIG. 1, so that the gas stream 90 emerges from the nozzle 106′ in the form of a plasma jet. When the generation unit 88′ for the apparatus 72 of FIG. 3 is used, the supply line 108′ is connected to the recirculation duct system 98, so that the gas stream 90 extracted from the treatment chamber 76 is supplied back to the nozzle 106′. The use of discharge means to produce a high frequency, high voltage discharge results in a gas stream 90 containing reactive species, but with low ozone concentration, which may reduce ozone exposure to users or the environment.

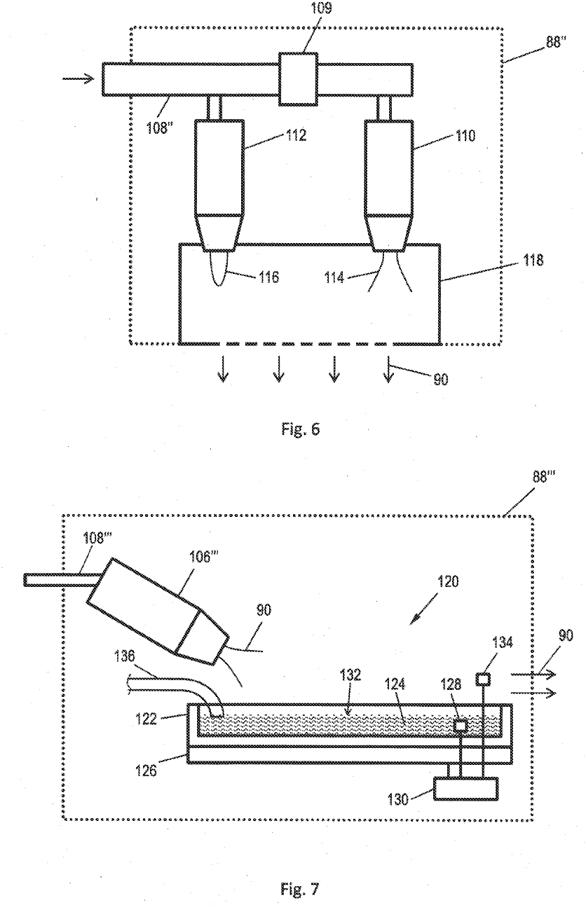

FIG. 6 shows a further alternative generation unit 88″ which can be used instead of the generation unit 88 for the apparatus of FIG. 3. The generation unit 88″ comprises a first part generation unit 110 and a second part generation unit 112, which in the present example are supplied with a respective partial gas stream via a common supply line 108″. If the part generation units 110, 112 require different gas flow rates for operation, the partial generation unit 110 with the higher gas flow rate can branch off from the supply line 108″ upstream of the part generation unit 112 with the lower gas flow rate, as shown in FIG. 6, and a throttle valve 109 can be provided upstream of the part generation unit 112.

When the generation unit 88″ is used for the apparatus 72 of FIG. 3, the supply line 108′ is connected to the recirculation duct system 98 so that a respective portion of the gas stream 90 exhausted from the treatment chamber 76 is supplied to the first and second part generation units 110, 112 as a respective partial gas stream.

The first part generation unit 110 comprises first discharge means for generating a dielectric barrier discharge in the first partial gas stream and can be designed in particular like the nozzle 32 of FIG. 2. The second part generation unit 112 comprises second discharge means for generating a high-frequency, high-voltage discharge in the second partial gas stream and can be designed in particular like the plasma nozzle 2 of FIG. 1.

During operation, the first partial gas stream 114 enriched with reactive species exits the first part generation unit 110 and the second partial gas stream 116 enriched with reactive species exits the second part generation unit 110. The first and second partial gas streams 114, 116 are combined and mixed in a mixing chamber 118 so that the resulting common gas stream 90 containing reactive species exits the mixing chamber 118.

It was found that the high ozone content of the first partial gas stream 114 generated by the first part generation unit 110 can be significantly reduced by mixing it with the second partial gas stream 116 generated by the second part generation unit 112, resulting in a common gas stream 90 that continues to be enriched with reactive species with reduced ozone load for users and environment.

FIG. 7 shows another alternative generation unit 88‴, which can be used instead of the generation unit 88 for the apparatus of FIG. 3. The generation unit 88‴ comprises a nozzle 106‴, which can be designed, for example, like the nozzle 32 of FIG. 2 or the plasma nozzle 2 of FIG. 1. When the generation unit 88‴ is used for the apparatus 72 of FIG. 3, the supply line 108‴ is connected to the circulating duct system 98, so that the gas stream 90 extracted from the treatment chamber 76 is supplied to the nozzle 106‴ as working gas. Instead of the nozzle 106‴, a combination of two nozzles as in FIG. 6 with a mixing chamber 118 can also be used in the generation unit 88‴.

Further, humidifying means 120 are provided for humidifying the gas stream 90 exiting the nozzle 106″. The humidifying means 120 comprise a water trough 122, which is filled with water 124 during operation. Heating means 126, for example in the form of a heating plate, and a temperature sensor 128 for measuring the water temperature are provided on the water trough 122. By means of provided control means 130, the water 124 in the water trough 122 can be adjusted to a predetermined temperature in this way.

The water trough 122 is arranged such that the gas stream 90 exiting the nozzle 106″ is directed toward the water trough 122, thereby blowing over the water surface 132 of the water 124 in the water trough 122. This increases the relative humidity of the gas stream 90, thereby providing a better disinfection effect in the treatment chamber 76.

A humidity sensor 134 may further be provided to sense the relative humidity of the gas stream 90 blown over the water surface 132. In this way, the temperature of the water can be controlled, for example, to achieve a predetermined relative humidity, in particular in the range of 90 % RH -100 % RH, preferably 95 % RH - 99 % RH. With a relative humidity just below 100 % RH, undesirable condensation can be reduced or prevented.

A water supply line 136 may be provided to replenish the amount of water lost from the water trough 122 due to evaporation.

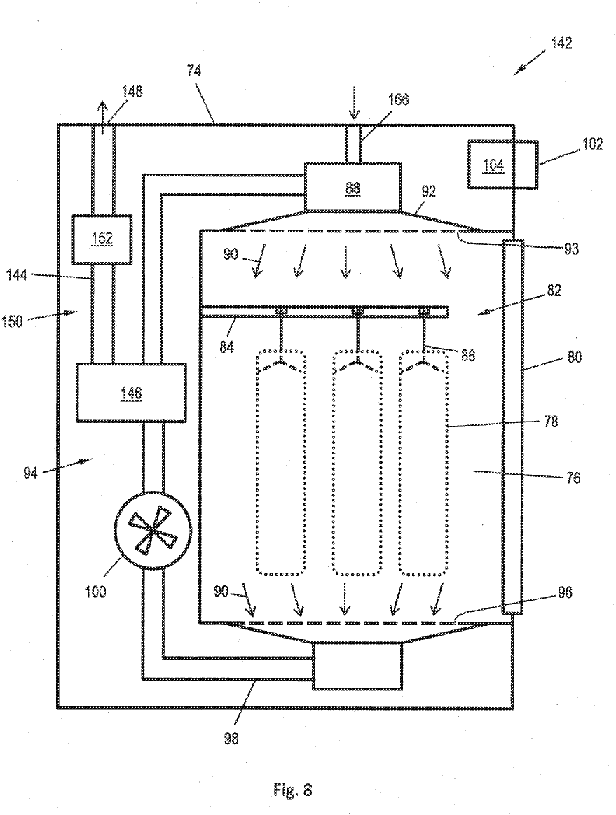

FIG. 8 shows a further exemplary embodiment of the apparatus for disinfecting objects or solids, preferably pieces of protective equipment, and for its use. The apparatus 142 in FIG. 8 has a similar structure to the apparatus 72 in FIG. 3. Corresponding components are therefore provided with the same reference numerals and reference is made in this respect to the corresponding description for FIG. 3.

The apparatus 142 differs from the apparatus 72 in that an outlet conduit 144 branches off from the recirculation duct system 98, through which the gas stream 90 carried in the recirculation duct system 98 is directed to an outlet opening 148 by switching a control flap 146. When the control flap is in the appropriate position, the openings on the floor side with the portion of the recirculation duct system and outlet conduit 144 connected thereto provide an outlet 150 provided at the treatment chamber 76 through which the gas stream 90 can be discharged from the treatment chamber 76. A neutralization device 152 is provided at the outlet 150, which is adapted to reduce the ozone content of the gas stream 90 discharged from the treatment chamber 76.

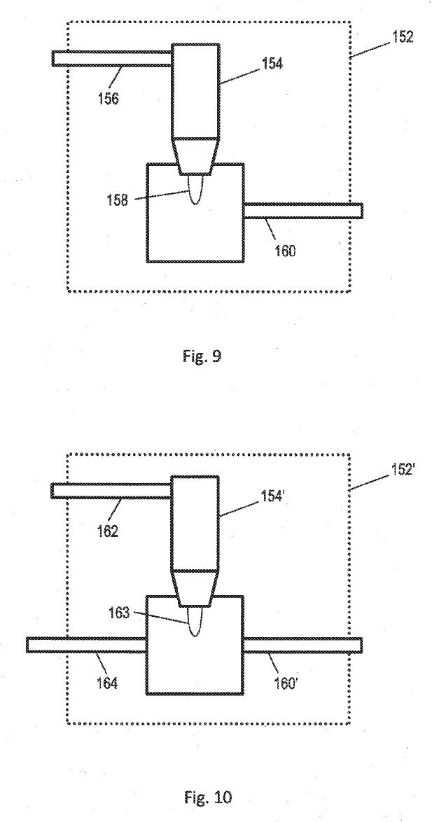

FIG. 9 shows the neutralization device 152 of the apparatus 142 of FIG. 8. The neutralization device 152 comprises a plasma nozzle 154 for generating an atmospheric plasma jet by high-frequency high-voltage discharges in a working gas. In particular, the plasma nozzle 154 may be configured like the plasma nozzle 2 shown in FIG. 2.

The plasma nozzle 154 is supplied with a working gas stream via a supply line 156, the supply line 156 being connected to the upstream portion of the outlet line 144 so that the plasma nozzle 154 is supplied with the gas stream 90 discharged from the treatment chamber 76 as a working gas stream, which then exits the plasma nozzle 154 in operation as a plasma jet 158. In this way, the ozone content in the gas stream 90 is significantly reduced. The gas stream 90 exiting the plasma nozzle 154 as the plasma jet 158 is then supplied to the downstream portion of the outlet line 144 via another line 160, thereby reaching the outlet port 148.

FIG. 10 shows an alternative neutralization device 152′ which can be used instead of the neutralization device 152 for the apparatus of FIG. 8. The neutralization device 152′ also has a plasma nozzle 154′, which can be designed in particular like the plasma nozzle 2 of FIG. 1. Unlike the neutralization device 152 in FIG. 9, the plasma nozzle 154′ is supplied with working gas via a separate working gas supply line 162, so that a plasma jet 163 emerges from the plasma nozzle 154′ during operation. Further, a supply line 164 connected to the upstream portion of the outlet line 144 is provided to supply the gas stream 90 exhausted from the treatment chamber 76 to the region of the plasma jet 163 so that the gas stream 90 is impinged with the plasma jet 163. This also results in a reduction of the ozone content in the gas stream 90. The gas stream 90 is then supplied to the downstream portion of the outlet conduit 144 via the further conduit 160′, thereby reaching the outlet port 148.

The neutralization device 152 or 152′ can be used, for example, at the end of a disinfection process to remove, neutralize and drain the gas stream 90 that is still partially enriched with reactive species, in particular ozone, from the treatment chamber 76, so that the ozone load for the user, when removing the pieces of protective equipment from the treatment chamber 76, and the environment is reduced.

Furthermore, a fresh air line 166, that can be switched in, may be provided through which fresh air can flow when the gas stream 90 is discharged from the treatment chamber 76. The fresh air line 166 may, for example, be connected to the generation unit 88, which is in particular no longer operated at the end of a disinfection process. The fresh air line 166 may also be arranged separately from the generation unit 88.

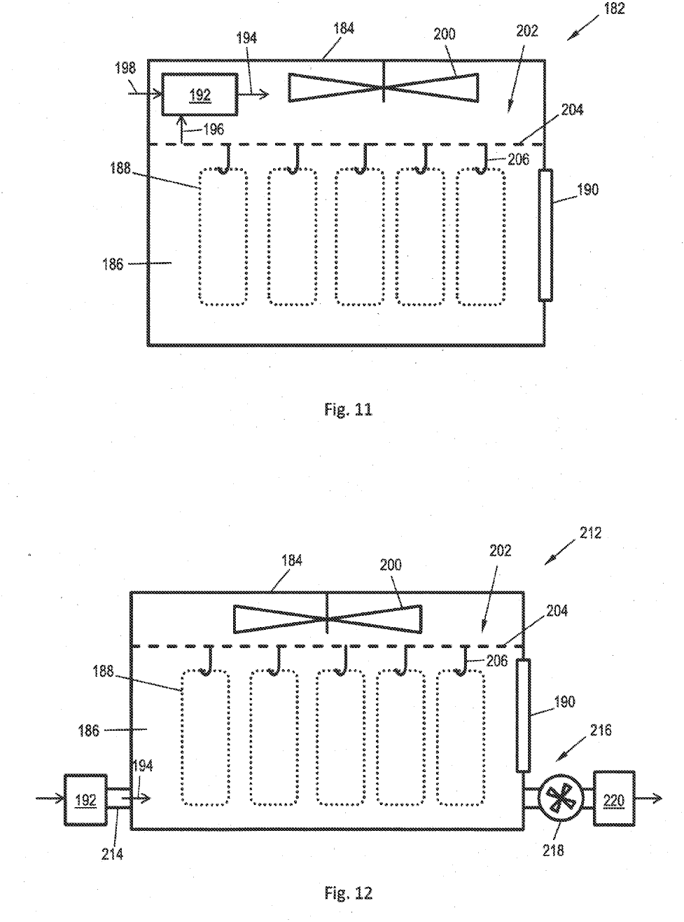

FIG. 11 shows another exemplary embodiment of the apparatus for disinfecting objects or solids, preferably pieces of protective equipment, and for its use. The apparatus 182 comprises a housing 184 in which is arranged a treatment chamber 186 for receiving one or more pieces of protective equipment 188, a closable airlock 190, for example in the form of a closable door or flap, through which pieces of protective equipment 188 can be introduced into and/or removed from the treatment chamber 186, and a generation unit 192 for generating reactive species in a gas stream, the generation unit 192 comprising discharge means configured to generate an electrical discharge in the gas stream.

In the apparatus 182, the generation unit 192 is arranged in the treatment chamber 186. As a result, the gas stream 194 which emerges from the generation unit 192 during operation and is enriched with reactive species passes directly into the treatment chamber 186.

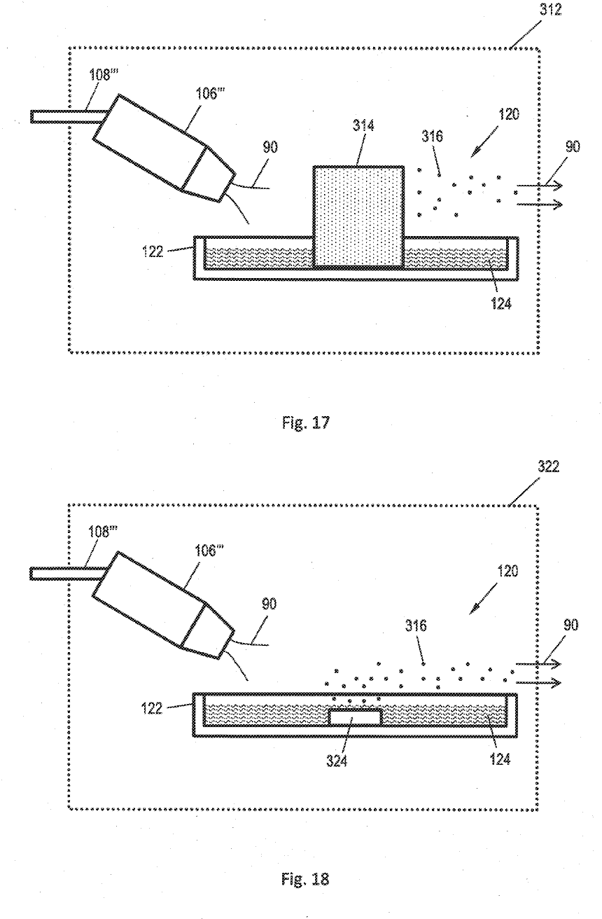

The generation unit 192 can be designed like one of the generation units 88, 88′, 88″ or 88‴ of FIGS. 4 - 7 or like one of the generation units 312 or 322 of FIGS. 17 - 18, whereby the supply line 108, 108′, 108″, 108‴ in the case of the apparatus 182 is not connected to a circulating air system as in the case of the apparatus 72, but to a supply line 196 from the treatment chamber 186 or to a separate supply line 198 for a working gas.

To better distribute the gas stream 194 containing the reactive species within the treatment chamber 186, a fan 200 is preferably disposed within the treatment chamber 186.

Furthermore, positioning devices 202 are provided in the treatment chamber 186 for positioning pieces of protective equipment 188 at predetermined locations in the treatment chamber 186. The positioning devices 202 are designed in the present case as a holder 204 with hooks 206 for suspending pieces of protective equipment 188 such as pieces of protective clothing or respiratory masks.

By arranging the generating unit 192 within the treatment chamber 186, a high disinfection effect can be achieved.

FIG. 12 shows another exemplary embodiment of the apparatus for disinfecting objects or solids, preferably pieces of protective equipment, and for its use. The apparatus 212 has a similar structure to the apparatus 182. Components corresponding to one another are provided with the same reference numerals and reference is made to the above description of FIG. 11.

The apparatus 212 differs from the apparatus 182 in that the generation unit 192 is not located inside but outside the treatment chamber 186 and is connected thereto via a supply line 214, so that the gas stream 194 containing the reactive species enters the treatment chamber 186. Further, an outlet 216 is provided at the treatment chamber with exhaust means in the form of a fan 218 for exhausting the gas stream 194 from the treatment chamber 186 after it has passed through the treatment chamber 186 or at the end of a disinfection process. A neutralization device 220 may also be provided at the outlet 216, which is configured to reduce the ozone content of the gas stream exhausted from the treatment chamber. The neutralization device 220 may be configured, for example, like the neutralization device 152 of FIG. 9 or like the neutralization device 152′ of FIG. 10.

By locating the generation unit 192 outside the treatment chamber 186, a longer life of the generation unit 192 can be achieved.

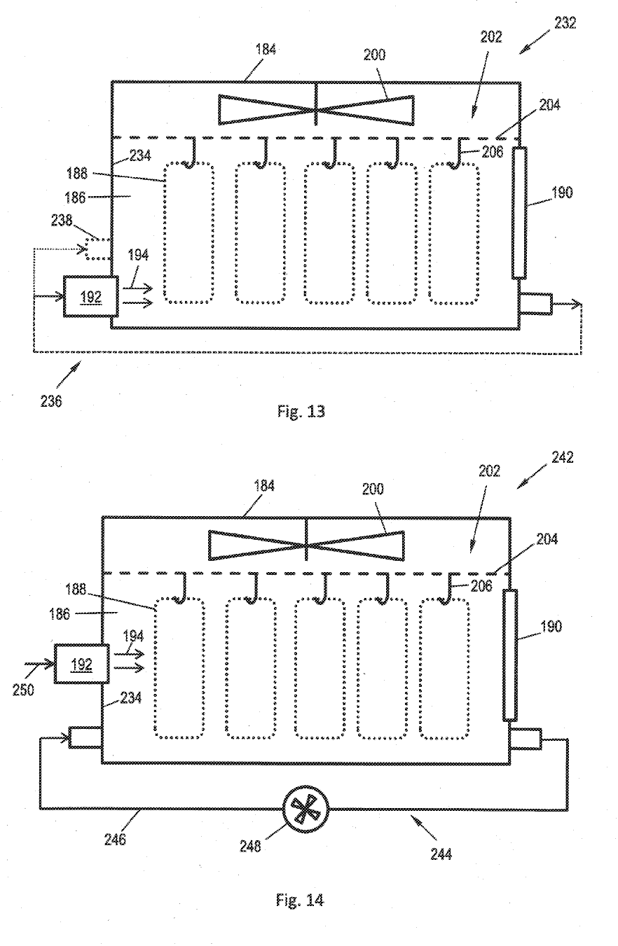

FIG. 13 shows another exemplary embodiment of the apparatus for disinfecting objects or solids, preferably pieces of protective equipment, and for its use. The apparatus 232 has a similar structure to the apparatus 182. Components corresponding to one another are provided with the same reference numerals and reference is made to the above description for FIG. 11.

The apparatus 232 differs from the apparatus 182 in that the generation unit 192 is integrated into the wall 234 of the treatment chamber 186. In this way, a good disinfection effect can be achieved while maintaining a good service life of the generation unit 192.

In one embodiment, the apparatus 232 may also be formed with a recirculation system 236. The gas stream directed through the recirculation system 236 may be supplied entirely to the generation unit 192. In an alternative embodiment, the gas stream directed through the recirculation system 236 is only partially supplied to the generation unit 192, while the remaining gas stream is directed directly into the treatment chamber 186 through a parallel recirculation outlet 238. In this way, the flow rate of the recirculation system 236 can be selected to be higher than the maximum flow rate of the generation unit 192, thereby achieving a better distribution of the gas stream enriched with the reactive species within the treatment chamber 186.

FIG. 14 shows another exemplary embodiment of the apparatus for disinfecting objects or solids, preferably pieces of protective equipment, and for its use. The apparatus 242 has a similar structure to the apparatus 182. Components corresponding to one another are provided with the same reference numerals and reference is made to the above description for FIG. 11.

The apparatus 242 differs from the apparatus 182 in that a recirculation system 244 is provided, which is arranged to discharge the gas stream from the treatment chamber 186 and to return it to the treatment chamber 186 via a recirculation duct system 246. For this purpose, the recirculation system particularly comprises a fan 248. The generation unit 192 is designed separately from the recirculation system and can be integrated into a wall 234 of the treatment chamber 186, as shown in FIG. 14, or alternatively can be arranged outside the treatment chamber 186 and connected thereto via a supply line. In a further embodiment, the generation unit 192 may also be arranged within the treatment chamber 186.

In particular, the generation unit 192 is supplied with working gas via a separate working gas supply 250 such that a gas stream 194 enriched with reactive species exits the generation unit 192. By separating the recirculation system 244 and the generation unit 192, the service life of the generation unit 192 can be extended, since the generation unit 192 is subject to less wear due to the separate working gas supply 250 than if a gas stream already or still enriched with reactive species is supplied to it as working gas.

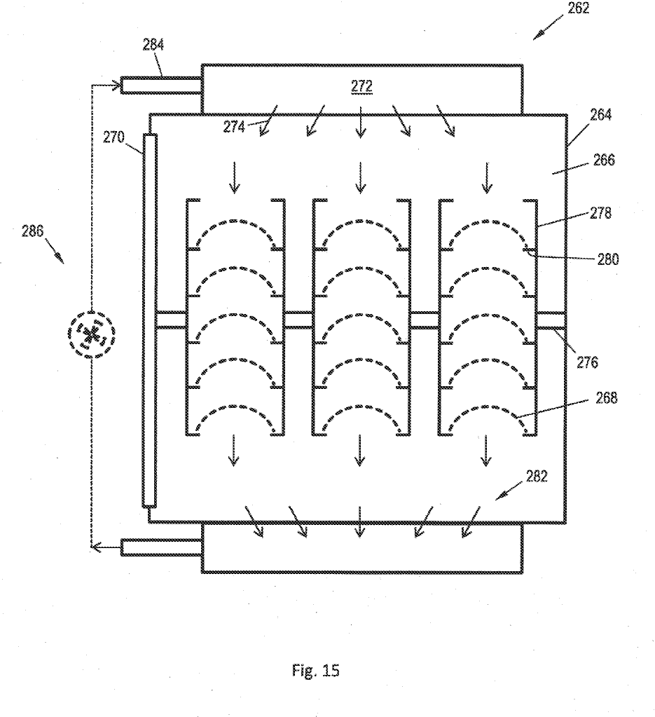

FIG. 15 shows another exemplary embodiment of the apparatus for disinfecting objects or solids, preferably pieces of protective equipment, and for its use. The apparatus 262 comprises a housing 264 having a treatment chamber 266 for receiving one or more pieces of protective equipment 268, a closable airlock 270 through which pieces of protective equipment 268 can be introduced into and/or removed from the treatment chamber 266, and a generation unit 272 for generating reactive species in a gas stream 274, the generation unit being arranged such that, during operation, the gas stream 274 enters the treatment chamber 266. The generation unit 272 includes discharge means arranged to generate an electrical discharge in the gas stream. In particular, the generation unit 272 may be configured like one of the generation units 88, 88′, 88″ or 88‴ of FIGS. 4 - 7 or like one of the generation units 312 or 322 of FIGS. 17 - 18.

A plurality of tubular elements 278 are disposed in the treatment chamber 266 via a support 276 such that, during operation, gas stream 274 introduced into the treatment chamber 266 flows through the tubular elements 278.

The tubular elements 278 are each configured to receive a plurality of breathing masks 268 one after another such that the breathing masks 268 are flowed through in succession as the gas stream 274 flows through the tubular element 278. To this end, the tubular elements 278 include retaining elements 280 arranged one after another for positioning the breathing masks 268. As an alternative to a plurality of holding elements 280 for individual breathing masks, for example, a holding element may be provided on which a stack of nested breathing masks is positioned.

In this manner, forced flow of the gas stream 274 enriched with reactive species through the breathing masks 268 is achieved, thereby effectively disinfecting the breathing masks 268.

Tubular members 278 represent positioning devices for positioning protective equipment, namely breathing masks, at predetermined locations within treatment chamber 266.

After flowing through the treatment chamber 266, the gas stream 274 may be directed out of the treatment chamber 266 through openings 282 in the wall thereof.

The supply line 284 of the generation unit 272 may be connected to a recirculation system 286 as in the apparatus 72 of FIG. 3. Alternatively, the supply line 284 may be a separate supply line for working gas. In this case, the openings 282 may form part of an outlet, as in the apparatus 212 of FIG. 12, to allow the gas stream 274 to exit the treatment chamber 266.

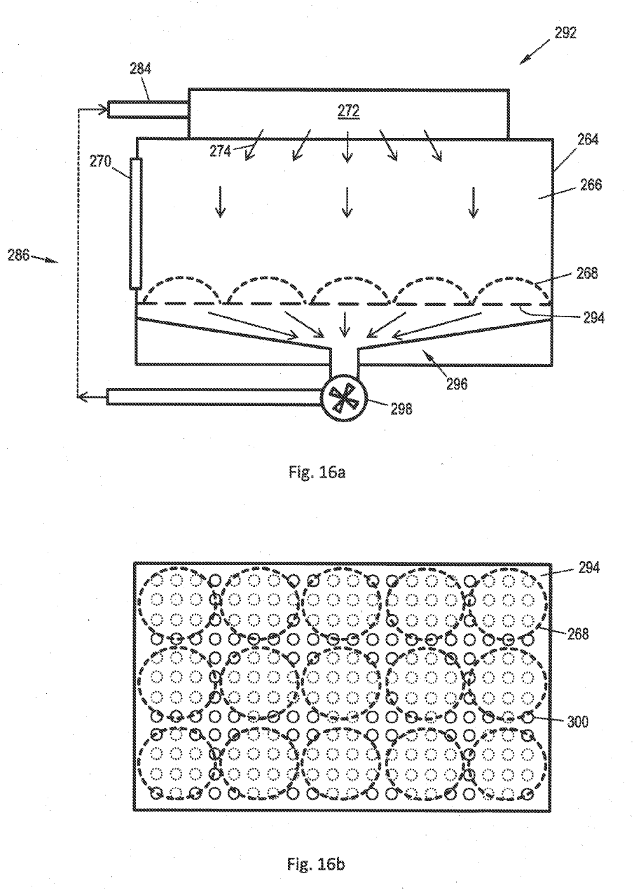

FIG. 16a shows a further exemplary embodiment of the apparatus for disinfecting objects or solids, preferably pieces of protective equipment, and for its use. The apparatus 292 has a similar structure to the apparatus 262 of FIG. 15, with mutually corresponding components being provided with the same reference numerals and in this respect reference is made to the above description for FIG. 15.

The apparatus 292 differs from the apparatus 262 of FIG. 15 in that a perforated plate 294 is provided as a positioning device instead of the tubular elements 278. Below the perforated plate 294, an exhaust 296 with a fan 298 is provided to draw the gas stream 274 through the holes 300 of the perforated plate 294.

To disinfect breathing masks 264, the user can arrange them on the perforated plate 294 as shown in FIGS. 16a-b. FIG. 16b shows a top view of the perforated plate 294 with the breathing masks 264 arranged thereon.

During operation, the gas stream 274 enriched with reactive species by the generation unit 272 is drawn in by the exhaust 296 and is thereby passed through the material of the breathing masks 264 on its way to the holes 300 of the perforated plate 294 so that they are disinfected.

In FIG. 16a, the apparatus 292 has only one perforated plate 294. However, it is also conceivable to arrange several perforated plates one above the other, optionally with respective suction or with a common suction, so that a larger number of breathing masks 264 can be treated simultaneously in the apparatus 292.

FIG. 17 shows a further alternative generation unit 312 for the apparatus of FIG. 3, which can be used instead of the generation unit 88 for the apparatus of FIG. 3. The generation unit 312 has a similar structure to the generation unit 88‴ of FIG. 7, whereby corresponding components are provided with the same reference numerals and reference is made to the above description of FIG. 7 in this respect.

The generation unit 312 differs from the generation unit 88‴ in that an evaporation body 314 made of porous, in particular sponge-like material is arranged in the water trough 122. The evaporation body 314 protrudes out of the water 124 so that the gas stream 90 flows around or through it.

The evaporation body 314 has a very large surface area relative to its volume. When the water trough 122 is filled with water 124, the water 124 also enters the evaporative body 314 or is drawn into the evaporative body 314 by capillary forces, resulting in improved evaporative performance. In addition, if the gas stream 90 has sufficient flow, droplets 316 can be entrained by the evaporative body 314 so that the gas stream 90 is also humidified thereby.

Heating means 126, control means 130, and sensors 128, 134, as well as water supply line 136, are omitted in FIG. 17, but may alternatively be partially or fully provided.

FIG. 18 shows a further alternative generation unit 322 for the apparatus of FIG. 3, which can be used instead of the generation unit 88 for the apparatus of FIG. 3. The generation unit 322 has a similar structure to the generation unit 88‴ of FIG. 7, whereby corresponding components are provided with the same reference numerals and reference is made to the above description of FIG. 7 in this respect.

The generation unit 322 differs from the generation unit 88‴ in that an ultrasonic nebulizer 324 is disposed in the water trough 122. During operation, the ultrasonic nebulizer 324 nebulizes the water 124 into small droplets 316, which are then entrained by the gas stream 90, thereby moistening it.

Heating means 126, control means 130, and sensors 128, 134, as well as water supply line 136, are omitted in FIG. 18, but may alternatively be partially or fully provided.

Instead of water 124, the water trough 122 in the generation unit 88‴, 312 or 322 can also be filled with plasma-activated water to further improve the disinfection effect. The use of an evaporator 314 or nebulizer 324 is advantageous in this case because the reactive species contained in the plasma-activated water are more effectively delivered to the gas stream 90 by the formation of droplets 316 effected in these embodiments.

The apparatus and the use have been described previously by way of example on the basis of exemplary embodiments of the apparatus for disinfection, in particular for sterilization, of items of protective equipment. However, the apparatus can also be used for disinfecting, in particular sterilizing, other goods, in particular powders, seeds or foodstuffs, in particular vegetables, fruit, lettuce, nuts such as hazelnuts, almonds, pulses or spices such as pepper. For this purpose, the apparatus can be appropriately dimensioned for the respective goods and, for example, corresponding positioning devices can be provided for the goods concerned.

1. An apparatus for disinfecting, in particular sterilizing, objects or solids, preferably pieces of protective equipment, in particular protective masks or protective clothing,
with a treatment chamber for receiving one or more objects or solids, preferably one or more pieces of protective equipment,
with a closable airlock through which objects or solids, preferably pieces of protective equipment, can be introduced into the treatment chamber and/or removed from the treatment chamber, and
with a generation unit for generating reactive species in a gas stream,
wherein the generating unit comprises discharge means configured to generate an electrical discharge in the gas stream and
wherein the generating unit is arranged such that the gas stream during operation, passes from the generating unit into the treatment chamber wherein
the discharge means are configured to generate an arc-like discharge in a gas stream, the arc-like discarge being generated by applying a high-frequency high voltage between electrodes.

2. (canceled)

3. (canceled)

4. The apparatus according to claim 1,
wherein the generating unit comprises a first part generating unit and a second part generating unit, wherein the first part generation unit comprises first discharge means for generating a dielectric barrier discharge in a first partial gas stream and wherein the second part generation unit comprises second discharge means for generating a high-frequency high-voltage discharge in a second partial gas stream.

5. The apparatus according to claim 1,
wherein the generation unit is configured to combine, in particular mix, the first partial gas stream and the second partial gas stream.

6. The apparatus according to claim 1,
wherein the generating unit is arranged within the treatment chamber.

7. The apparatus according to claim 1,
wherein the generating unit is integrated into the wall of the treatment chamber.

8. The apparatus according to claim 1,
wherein the generating unit is arranged outside the treatment chamber and is connected to the treatment chamber in such a way that the gas stream enters the treatment chamber operation.

9. The apparatus according to claim 1,
wherein a fan is arranged in the treatment chamber.

10. The apparatus according to claim 1,
wherein one or more positioning devices are arranged in the treatment chamber for positioning objects or solids, preferably pieces of protective equipment at a predetermined position in the treatment chamber.

11. The apparatus according to claim 1,
a tubular element is arranged in the treatment chamber in such a way that, during operation, the gas stream introduced into the treatment chamber flows through it, the tubular element being configured to receive a plurality of objects, preferably a plurality of pieces of protective equipment in particular a plurality of breathing masks, in such a way that the objects, preferably pieces of protective equipment, in particular the breathing masks, are flowed through by the gas stream, preferably one after the other, when the gas stream flows through the tubular element.

12. The apparatus according to claim 1,
wherein an outlet is provided on the treatment chamber for discharging the gas stream from the treatment chamber.

13. The apparatus according to claim 12, wherein extraction means are connected to the outlet and are configured to extract the gas stream from the treatment chamber.

14. The apparatus according to claim 12,
wherein a neutralization device is provided at the outlet and configured to reduce the ozone content of the gas stream .

15. The apparatus according to claim 14,
wherein the neutralization device comprises a plasma nozzle for generating an atmospheric plasma jet.

16. The apparatus according to claim 15,
wherein the plasma nozzle is configured to generate a plasma jet by high-frequency high-voltage discharges in a working gas stream.

17. The apparatus according to claim 15,
wherein the gas stream is passed through the plasma nozzle as a working gas stream.

18. The apparatus according to claim 15,
wherein the gas stream is impinged with the plasma jet emerging from the plasma nozzle.

19. The apparatus according to claim 1,
wherein a recirculation system is provided which is configured to discharge the gas stream from the treatment chamber and to resupply it to the treatment chamber via a recirculation duct system.

20. The apparatus according to claim 19,
wherein the recirculation system comprises a fan, in particular a side channel compressor, which fan is configured to extract the gas stream from the treatment chamber and to direct it through the recirculation duct system.

21. The apparatus according to claim 19, wherein the generation unit is integrated into the recirculatin system in such manner that the gas stream conducted through the recirculation system is at least partially supplied to the generating unit.

22. The apparatus according to claim 19,
wherein the generation unit is formed separately from the recirculation system.

23. The apparatus according to claim 1
wherein humidifying means are provided which are configured to increase the relative humidity of the gas stream and/or the relative humidity in the treatment, preferably to a relative humidity in the range of 90% RH - 100% RH, preferably 95% RH - 99% RH.

24. The apparatus according to claim 23,
wherein humidifying means comprise a preferably heatable water trough.

25. The apparatus according to claim 24,
wherein the water trough is arranged such that the gas stream from the generating unit is directed onto the water trough during operation.

26. The apparatus according to claim 24,
wherein an evaporation body and/or a nebulizer is arranged in the water trough.

27. The apparatus according to claim 1,
wherein a perforated plate is arranged in the treatment chamber in such a way that, during operation, the gas stream introduced into the treatment chamber flows through it, the perforated plate being arranged in such a way that a plurality of objects, preferably a plurality of pieces of protective equipment, in particular a plurality of breathing masks, can be positioned on the perforated plate.

28. A use of the apparatus according to claim 1 for disinfecting, in particular sterilizing, objects or solids, preferably pieces of protective equipment, in particular protective masks or protective clothing.
